(19) United States
(12) Patent Application Publication
(10) Pub. No.: US 2014/0037561 A1
(43) Pub. Date: Feb. 6, 2014

(54) COMPOSITION AND METHODS OF ENHANCED SKIN CELL TURNOVER

(71) Applicants: John E. Kulesza, Berlin, CT (US); Mark Rubin, Encino, CA (US)

(72) Inventors: John E. Kulesza, Berlin, CT (US); Mark Rubin, Encino, CA (US)

(21) Appl. No.: 13/983,464

(22) PCT Filed: Feb. 3, 2011
(86) PCT No.: PCT/US2011/023614

§ 371 Date: Oct. 21, 2013

(51) Int. Cl. A61K 8/36; A61K 8/49; A61Q 19/08; A61K 8/34

(52) U.S. Cl. CPC A61K 8/36; A61K 8/347; A61K 8/4953; A61Q 19/08
USPC 424/59; 514/557; 514/266.23; 514/458; 514/163; 514/529

(57) ABSTRACT

A composition is provided that capitalizes on the biological activity of trichloroacetic acid, but effectively reduces or eliminates TCA induced irritation allowing the composition to be used as a wrinkle effacer or stimulant of skin cell turnover; uses previously thought as commercially impossible with TCA. Also provided are processes of effacing wrinkles or stimulating skin cell turnover including applying the inventive composition to the skin of a subject.

### FIELD OF THE INVENTION

The invention relates to the prevention of skin ageing. Generally, compositions and methods are provided that enhance skin cell turnover, reduce wrinkles, and improve appearance in a user while reducing skin irritancy observed with traditional chemical peel agents.

### BACKGROUND OF THE INVENTION

Animal skin serves essential roles in protecting an organism from environmental insults. Numerous specific skin functions include protection, excretion, secretion, absorption, thermoregulation, pigmentogenesis, accumulation, sensory perception, and regulation of immunological processes. The structural and chemical changes in the skin during ageing reduce the effectiveness of skin to achieve each of these principal functions. Upon reaching the later stages of life most previously robust skin functions are reduced, some by as much as 50-60%. The physiological changes associated with these reductions include impairment of the barrier function, decreased turnover of epidermal cells, reduced numbers of keratinocytes and fibroblasts, and a reduced vascular network particularly around hair bulbs and glands. A side effect of these changes is an increase in the appearance of skin wrinkles and previously unrecognized pigmented areas.

In an effort to prevent unwanted ageing of skin or the appearance of aged skin, many people use chemical peels to reduce wrinkles and increase skin cell renewal. Normal skin represents a balanced cell renewal system where fully differentiated corneocytes are continually shed and replaced by new cells generated at lower skin levels. Normally this process occurs at a steady state whereby the new cells are produced at rates equivalent to the shedding of surface cells. Chemical peels offer a way to increase this rate of skin renewal. Peels function by actively removing damaged outer layers of skin, replacing it with new, more hydrated, and more robust skin cells. This improves the smoothness of skin, reducing wrinkles, and providing a younger appearance. Chemical peels are also beneficial for those with facial blemishes such as acne scarring, anomalous pigmentations, or for removing actinic keratoses.

Chemical peels are available in several formulations that provide superficial, moderate, or deep peels. Irritancy is a hallmark of all chemical peels, and all peels induce irritancy in the skin of a subject, even superficial peels. Traditionally, this irritancy is a source of the peeling nature of the compositions.

Superficial chemical peels typically remove only the upper corneous layer of the epidermis. Peeling agents include alpha-hydroxy acids including citric acid (e.g. citrus-derived), glycolic (derived from sugar cane), lactic acid (derived from milk), malic acid (derived from apples) and tartaric acid (derived from grapes). These are the mildest of agents yet produce extreme irritancy such as stinging, redness, dryness, and other skin irritation. Typically only mild skin smoothing or acne improvements are observed with this level of chemical peel.

Moderate depth chemical peels penetrate into the upper layers of the dermis causing more aggressing sloughing of the outer skin cell layers. Typical agents for moderate peels include higher concentration hydroxy acids and trichloroacetic acid at concentrations of 20-50%. The greater the concentration of the peeling agent, the deeper the peel. Moderate peels function to smooth fine surface wrinkles, surface blemishes to the skin, and unwanted pigmentation. The irritation to the skin is much greater than a superficial peel and recovery times and precautions are also increased.

The deep peel, commonly achieved by applications of phenol, is used to treat deep wrinkling, remove deeper pigmentation anomalies, and treat precancerous skin growths. Phenol peels contain highly irritating ingredients that can have long lasting affects on skin appearance. Phenol peels can leave the skin with a lighter pigmentation and damage the skin cells such that new pigment production cannot occur. Recovery from phenol peels can be several months and may require permanent avoidance of sun exposure.

The ubiquitous irritating nature of all prior art skin renewal enhancing compositions is essential for function and the primary source of unwanted side effects of treatment. Thus, there exists a need for compositions and methods of enhancing skin renewal while reducing irritancy.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

Trichloroacetic acid (TCA) is believed in the art to be unacceptable for daily or other regular application to the skin for purposes of exfoliating or wrinkle effacement due to the high irritancy experienced with TCA. (See e.g. Smith, W P., Cosmetics and Toiletries, 1994; 109:41-48.) Compositions are provided that reduce or eliminate the irritancy associated with prior art TCA compositions allowing TCA to be applied to the skin on a daily or more frequent basis and be well accepted by the subject.

Compositions for topical application are provided including trichloroacetic acid and a stabilant with a normal boiling point in excess of 50° C.; where the trichloroacetic acid and the stabilant are present in a ratio from 0.1:1 to 5:1. In some embodiments, TCA is present at 10% or less by weight, optionally at 5% or less. A stabilant is optionally propylene glycol, butylene glycol, ethoxydiglycol, benzyl alcohol, or glycerin.

An anti-irritant is optionally included that optionally includes one or more indolequinazoline alkaloids. The anti-irritant is optionally evodiox. An anti-irritant is optionally present at 0.1 percent to 5 percent by weight.

The compositions optionally include one or more bioactive agents. A bioactive agent is optionally: vitamin A or a derivative of vitamin A; vitamin E or a derivative of vitamin E; a hydroxy acid; benzoyl peroxide; resorcinol; an antimicrobial; an anti-neoplastic agent; an anti-viral agent; a nonsteroidal anti-inflammatory agent; a UV filter; or an immunomodulator. The bioactive agent is optionally present at a therapeutically effective amount which is readily determined by those of skill in the art. Illustratively, when a bioactive agent is vitamin A or its derivatives the vitamin A or its derivatives are present at between 0.001 to 2 weight percent. Optionally, a vitamin A derivative is: retinal; retinoic acid; retinyl ester; retinol; tretinoin; isotretinoin; adapalene; tazarotene; or combinations thereof. A bioactive agent is optionally salicylic acid, acetylsalicylic acid, or combinations thereof. A bioactive agent is optionally vitamin E or a derivative of vitamin E present at 0.1 to 10 weight percent.

An inventive composition optionally comprises one or more of formulas A to M or O to CC.

Also provided are processes of increasing skin cell renewal or increasing wrinkle effacement including applying any of the compositions described herein, or their equivalents, to the skin of a subject. The compositions are optionally applied daily, optionally nightly. The composition is optionally applied for a first period. A first period is optionally twenty days or more, optionally 6 weeks or more. A second composition with a differing amount of TCA is optionally applied for a second period that may be prior to, simultaneous with, or subsequent to a first period. A second composition optionally has more or less TCA than a first composition.

Also provided are processes of preventing decarboxylation of trichloroacetic acid in water including combining trichloroacetic acid with water, and further combining a stabilant with a normal boiling point in excess of 50° C.

### BRIEF DESCRIPTION OF THE DRAWINGS

FIG. 1 is a photograph of a subject prior to (A) and following 12 weeks (B) of daily administration of two embodiments of an inventive composition;

FIG. 2 represents quantitative measurements of wrinkle reduction in the skin of a subject before and following 12 weeks of daily administration of two embodiments of an inventive composition.

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

The invention has utility as a composition for enhancing skin renewal with greatly reduced irritancy. The invention includes trichloroacetic acid (TCA) that is stabilized in an aqueous solution by the addition of a stabilant with a boiling point in excess of 50° C. and where the ratio of TCA:stabilant is at or anywhere in the range of 0.1:1 to 5:1. The resulting topical composition is suitable for application to the human skin, optionally for the treatment or prevention of wrinkles, dry skin, dermatoses, acne, keratoses, photo-aging, melasma, itching, inflammation, pseudofolliculitis barbae (razor bumps) and other skin conditions treatable by increased skin cell renewal.

TCA is commonly used for chemical peels due to its ability to affect skin turnover rates. The concentration of TCA in chemical peels is typically 20% or greater owing to the high level of irritancy of TCA as well as its lack of stability low concentrations. The high level of irritancy observed at concentrations less than 20% historically made TCA inappropriate for superficial skin peels or general topical application. Other active agents such as the hydroxy acids are considered in the art to be much more desirable because of the greatly reduced irritancy associated with these compounds. TCA is considered in the art to be of little cosmetic value due to its high irritancy, even at concentrations as low as 0.5%. (See Smith, W P, Cosmetics and Toiletries, 1994; 109:41-48.)

The present invention proceeds contrary to the established knowledge in the art and provides a composition with relatively low concentration of TCA that is present in a non-volatile stabilant, the combination of which produces greatly reduced irritancy than the hydroxy acids preferred in the art for superficial peels or consumer applied topical medicaments. Some embodiments of the invention also include an anti-irritant including one or more indolequinazoline alkaloids.

TCA is an analogue of acetic acid with each of the hydrogens on a methyl group replaced by chlorine. TCA is made by mechanisms known in the art and is readily available from a variety of sources illustratively including Sigma-Aldrich, St. Louis, Mo. Some embodiments of the invention include TCA at concentrations at or less than 10% by weight. The TCA concentration will depend upon the manner in which the composition is used. Compositions intended to be dispensed by physicians or other trained professionals generally include relatively high acid concentrations since these materials will be applied therapeutically, in a controlled setting, and for restricted periods of time. Compositions for home use are optionally of lower TCA concentration (e.g. 0.1 to 8% by weight).

Examples of TCA concentrations range from 0.1 to 10% by weight including any value or range therebetween. Optionally, TCA is present at less than 10% by weight, optionally less than 5% by weight. Some embodiments include TCA concentrations at 0.5, 1, 2, 3, 4, 5, 6, 7, 8, 9, or 10% by weight. It is appreciated that TCA is optionally present at concentrations suitable for use as a chemical peeling agent. Such concentrations are optionally in excess of 10%, optionally 15%, 20%, 25%, 30%, 40%, 50%, or any value or range between 10% and 50%. The present invention provides a TCA based composition suitable for use to induce a chemical peel in a subject with far fewer irritating side effects and increased recovery time.

A composition includes a stabilant for the TCA. Optionally, a stabilant has a normal boiling point in excess of 50° C., 75° C., 100° C., 150° C., 175° C., 200° C., 250° C., 300° C., or 350° C. In some embodiments, a stabilant has a normal boiling point in excess of 500° C. Optionally, a stabilant has a normal boiling point between 50° C. and 500° C.

A stabilant is soluble in water at or above the concentrations relative to TCA of the inventive compositions. As solubility values are measured in weight solute per weight or volume of solvent, the ranges of solubility values vary with the stabilant used. One of ordinary skill in the art can readily determine solubility of a stabilant in water. A stabilant is optionally miscible in water.

A stabilant is optionally suitable for use in a composition suitable for topical administration to a subject. As such, it is appreciated that a stabilant is optionally non-toxic to the subject. In some embodiments, a stabilant is an alcohol, optionally with a contiguous hydrocarbon chain of between three and 6 carbons. Illustrative examples of stabilants include ethanol, ethoxydiglycol, propylene glycol, 1,3-propanediol, butylene glycol (1,3-butanediol), 1,2-butanediol, 1,4-butanediol, 2,3-butanediol, benzyl alcohol, isopropanol, ketones such as acetone, and glycerin.

It is appreciated that more than one organic stabilant is optionally present in an inventive composition. In some embodiments, 2, 3, 4, 5, 6 or more organic stabilants are present. The ratio of weight percent of the TCA to the organic stabilant is optionally from 0.1:1 to 5:1, optionally, 0.5:1 to 2:1. In some embodiments, the weight percent of TCA and stabilant are equal. Optionally, the stabilant is present at a lower weight percent than the TCA.

A stabilant is optionally glycerin. Sources of glycerin are known in the art such as Dow Chemical Co. Glycerin operable herein is optionally anhydrous, or contains less than 5 percent moisture. Optionally, glycerin is a 96% USP glycerin. Optionally, glycerin is a 99.5% USP glycerin. In some embodiments the glycerin is dehydrated such as by vacuum distillation or pervaperation such as that described by Khairnar and Pangarkar, J. Am. Oil Chem. Soc., 2004; 81:505-10.

An inventive composition is optionally an aqueous solution of TCA. An aqueous solution is optionally at least 60% water. Optionally, water is present at a weight percent in excess of 40%, 45%, 50%, 55%, 60%, 65%, 70%, 80%, 85%, 90%, 91%, 92%, 93%, 94%, 95%, 96%, 97%, or any value or range therebetween. Water is optionally purified so as to remove contaminants such as solids and other microorganisms, or subjected to processes to remove contaminating ions. Illustratively, compositions include deionized water prepared by methods and using apparatuses known in the art. Methods of purifying or filtering water are well known in the art.

An inventive composition optionally includes one or more anti-irritants. An anti-irritant is any molecule or combination of molecules, naturally derived or synthetic, that will reduce or prevent irritation to the skin of a subject. In some embodiments an anti-irritant is naturally derived such as a plant extract, or plant or animal derived molecule. Optionally, an anti-irritant is synthetic and derived from a non-natural source. In some embodiments, an anti-irritant is derived from a combination of sources, optionally natural and synthetic.

An anti-irritant is optionally present in an inventive composition in an amount ranging from 0.1% to 5% by weight, or any value or range therebetween. Optionally, an anti-irritant is present in an amount from 0.5% to 3%. In some embodiments, an anti-irritant is present at 0.5%, 0.75%, 1.0%, 1.5%, or 2% by weight. It is appreciated that more than 5% by weight of a composition may be an anti-irritant.

Illustrative examples of anti-irritants include: plant extracts; plant oils; buffering agents; lipids such as ceramides, fatty acids, and fatty alcohols; and steroids such as cholesterol. Illustrative examples of anti-irritants are plant oils, illustratively lemon oil. The inclusion of lemon oil in a TCA based composition is described in European Patent EP 0 644 752 B1. Other plant oils may be included in a composition illustratively including agar oil, ajwain oil, angelica root oil, anise oil, balsam oil, basil oil, bergamot oil, black pepper essential oil, buchu oil, cannabis flower essential oil, caraway oil, cardamom seed oil, carrot seed oil, cedarwood oil, chamomile oil, cinnamon oil, cistus, citronella oil, clary sage, clove leaf oil, coriander, costmary oil, cranberry seed oil, cumin oil/Black seed oil, cypress, davana oil, dill oil, eucalyptus oil, fennel seed oil, fenugreek oil, frankincense oil, galbanum, geranium oil, ginger oil, grapefruit oil, grape seed oil (e.g. Vitis vinifera), henna oil, jasmine oil, juniper berry oil, lavender oil, lemon oil, lemongrass oil, litsea cubeba oil, melissa oil (Lemon balm), mentha arvensis oil/mint oil, mugwort oil, mustard oil, myrrh oil, neroli oil, orange oil, oregano oil, orris oil, parsley oil, patchouli oil, perilla essential oil, pennyroyal oil, peppermint oil, pine oil, rose oil, rosehip oil, rosemary oil, rosewood oil, sassafras oil, savory oil, schisandra oil, spearmint oil, star anise oil, tarragon oil, tea tree oil, thyme oil, vetiver oil, yarrow oil and ylang-ylang oil.

Methods of producing plant oils are known in the art illustratively by oil extraction or by pressing the seeds of a plant such as grape seeds, peanut seeds, or other seed. Many plant oils are available in the market and a person of ordinary skill in the art recognizes where to obtain them.

Unlike the plant oils that are expected to provide anti-irritant effects by solvating, emulsifying, or sequestering the TCA to moderate its effects, the inventors found that the full activity of TCA can be maintained and the expected irritation reduced by the addition of a plant extract that includes at least one of dehydroevodiamine, evodine, evodiamine, or rutaecarpine. An extract produced from the Evodia rutaecarpa fruit, optionally known as Wu-Chu-Yu, is optionally added as an anti-irritant including the above ingredients. Evodia rutaecarpa fruit extract contains many organic compounds that have been studied individually, and various effects have been attributed to the individual components, the major ones being the indolequinazoline alkaloids, also known as quinazolindocarboline alkaloids. In Evodia fruit the primary indolequinazoline alkaloids are rutaecarpine, evodiamine and dehydroevodiamine. Other components of the Evodia rutaecarpa fruit extract include those listed by Xu H Y et al., Chinese Herbal Medicines, 2010; 2(2):112-117.

In some embodiments, an Evodia rutaecarpa fruit extract is sold under the tradename EVODIOX. EVODIOX, sold by Barnet Products Corp., Englewood Cliffs, N.J., is a mixture of Evodia rutaecarpa fruit extract, butylated hydroxytoluene, butylene glycol, and phenoxyethanol. When the term “evodiox” is used herein, designates a mixture of Evodia rutaecarpa fruit extract, butylated hydroxytoluene, butylene glycol, and phenoxyethanol in proportions equal to the material sold as EVODIOX.

Methods of producing plant extracts are known in the art and illustratively include grinding of plant material and extraction in water, ethanol, or combinations. To improve the extract quality, an extraction is optionally performed with heating to between 50° C. to 100° C. Extracted material is optionally filtered one or more times prior to evaporation of extraction solvent. It is appreciated that other methods of preparing plant extracts are known in the art and applicable to the preparation of one or more plant extracts useful in the invention.

Other specific illustrative examples of anti-irritants include licorice and its extracts, dipotassium glycyrrhizinate, oat and oat extracts, candelilla wax, alpha bisabolol, aloe vera, Manjistha (extracted from plants in the genus Rubia, particularly Rubia cordifolial), and Guggal (extracted from plants in the genus Commiphora, particularly Commiphora Mukul). It is appreciated that more than one anti-irritant may be included in an inventive composition. In some embodiments, evodiox is included along with a second, third, or additional anti-irritant.

An inventive composition optionally includes one or more bioactive agents. A “bioactive agent” is a chemical or biological molecule or combination of molecules suitable for delivery to the skin of a subject. Optionally, an active agent has pharmaceutical activity and is present for the treatment or prevention of a skin condition. A bioactive agent optionally has activity that is: anti-inflammatory; anti-bacterial; anti-parasitic; anti-viral; analgesic; immunity modulation and/or stress relaxant properties; alters cell growth or death; promotes apoptosis; induces alterations in DNA such as alkylation, oxidation, or base hydrolysis; inhibits or stimulates enzyme activity; or combinations thereof. Although TCA has properties of a bioactive agent, the term “bioactive agent” is exclusive of TCA.

Bioactive agents include but are not limited to active herbal extracts; acaricides; age spot and keratose removing agents; allergens; analgesics; local anesthetics; antiacne agents; antiallergic agents; antiaging agents; antibacterials; antibiotics; antiburn agents; antineoplastic and/or ophthalmic agents illustratively including 5-fluorouracil, doxorubicin, imiquimod, and sodium [o-(2,6-dichloranilino) phenyl] acetate; antidandruff agents; antidepressants; antidermatitis agents; antiedemics; antihistamines; antihelminths; antihyperkeratolyte agents; antiinflammatory agents such as steroidal or non-steroidal anti-inflammatory agents illustratively flurbiprofen, ibuprofen, naproxen, indomethacin, glucocorticoids such as hydrocortisone, and other anti-inflammatory compounds; antilipemics; antimicrobials such as azelaic acid, erythromycin, sodium sulfacetamide, tetracycline and derivatives, and clindamycin; anti-mitotic drugs illustratively colchicine taxol and related compounds; antiproliferative agents; antioxidants; anti-wrinkle agents; antipruritics; antipsoriatic agents; antirosacea agents; antiseborrheic agents; antiseptics; antiswelling agents; antiviral agents illustratively ganciclovir, trifluorothymidine and related compounds; antiyeast agents; astringents; aromatic molecules such as benzoyl peroxide, resorcinol, hydroquinone; topical cardiovascular agents; chemotherapeutic agents; corticosteroids; dicarboxylic acids; disinfectants; fungicides; hair growth regulators; skin growth factors illustratively TGFβ, epidermal growth factor, platelet derived growth factor, granulocyte macrophage colony stimulating factor (GM-CSF), interleukins, and others typically used for dermatological therapies; hormones; hydroxy acids; immunomodulators such as immunosuppressants and immunoregulating agents; insecticides; insect repellents; keratolytic agents; lactams; metals; metal oxides; miticides; neuropeptides; non-steroidal anti-inflammatory agents; oxidizing agents; pediculicides; photodynamic therapy agents; sanatives; scabicides; self tanning agents; skin whitening agents; vasoconstrictors; vasodilators; vitamins such as vitamin A or its derivatives, or vitamin D or its derivatives, or vitamin E or its derivatives; wound healing agents; wart removers; drugs that act on actin polymerization illustratively phalloidin, cytochlasin B and related compounds; inhibitors of dihydropyrimidine dehydrogenase (DPD), thymidine phosphorylase (TP) and/or uridine phosphorylase (UP) enzyme inhibitors; ultraviolet light (UV) filters illustratively benzophenone derivatives such as oxybenzone, octocrylene, octyl methoxycinnamate, and avobenzone; radiation proactive agents illustratively methyluracils such as 6-methyluracil and 4-methyluracil; and immunomodulating molecules such as tacrolimus, and pimecrolimus. As is known to one of skill in the art, in some instances a specific bioactive agent may have more than one activity, function or effect.

Optionally, a bioactive agent is vitamin A or its derivatives. Examples of vitamin A or its derivatives illustratively include retinoids such as retinal, retinoic acid, retinyl ester, retinol, tretinoin, isotretinoin, adapalene, tazarotene, and the like. In some embodiments a vitamin A or its derivatives are present at between 0.001 to 2 weight percent.

A bioactive agent is optionally vitamin E or its derivatives. Illustrative derivatives of vitamin E include sodium vitamin E phosphate, lauryl imino dipropionic acid tocopheryl phosphate, tocopheryl glucoside, tocopheryl succinate, tocophersolan (tocopheryl polyethylene glycol 1000 succinate), tocophereth-5, tocophereth-10, tocophereth-12, tocophereth-18, or tocophereth-50. Vitamin E or its derivatives are optionally present in an amount ranging from 0.1% to 10% by weight, or any value or range therebetween. The use of vitamin E or its derivatives to increase exfoliation are illustrated in U.S. Pat. No. 6,645,514.

Examples of hydroxy acids illustratively include beta hydroxy acids such as salicylic acid, acetylsalicylic acid, and the like.

Numerous skin or systemic conditions are treatable with the inventive composition illustratively including acne, wrinkles, dryness, eczema, psoriasis, actinic and nonactinic keratoses, rosaceous, among others. U.S. Pat. No. 3,932,665 describes retinal as a therapeutic agent in a method for treating acne by topical application. The topical administration of 5-fluorouracil for treatment of keratoses is described in U.S. Pat. No. 4,034,114.

The compositions of the invention optionally include 0.005 to 1.0 weight percent retinol, in which case they are optionally applied directly to the skin, or supplied as more concentrated solution containing higher levels of active agent, in which case prior to application they are diluted optionally by means of a cosmetically acceptable carrier to a desired level such as 0.005 to 1.0 weight percent for retinol.

Optionally, a bioactive agent is present in less than 30 percent w/w amounts. Optionally, a bioactive agent is present at a weight percent of 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.5, 0.1, 0.01, 0.001, 0.0001, any level in between or any range therein. Optionally, a bioactive agent is present at 20 percent w/w. Illustratively, when azelaic acid is an active agent it is present at 15 to 25 percent w/w. A vitamin A derivative is optionally present at 0.001 to 2 percent by weight. Imiquimod is optionally present at 3 to 8 percent by weight. Benzoyl peroxide is optionally present at 1 to 10 percent by weight. It is within the skill of the art to determine the optimal level of active agent in either a concentrated solution or a final solution for application.

In some embodiments, the bioactive agent is an anti-infective agent. Illustrative examples of an anti-infective agent include an antibiotic agent, an antibacterial agent, an antifungal agent, an agent that controls yeast, an antiviral agent and an antiparasitic agent. Exemplary anti-infective agents are beta-lactam antibiotic, an aminoglycoside, an ansa-type antibiotic, an anthraquinone, an azole, metronidazole, an antibiotic glycopeptide, a macrolide, erythromycin, clindamycin, an antibiotic nucleoside, an antibiotic peptide, polymyxin B, an antibiotic polyene, an antibiotic polyether, an antibiotic quinolone, an antibiotic steroid, fucidic acid, mupirocin, chloramphenicol, a sulfonamide, tetracycline, an antibiotic metal, silver, copper, zinc, mercury, tin, lead, bismuth, cadmium, chromium, an oxidizing agent, iodine, iodate, a periodate, a hypochlorite, a permanganate, a substance that releases free radicals and/or active oxygen, a cationic antimicrobial agent, a quaternary ammonium compound, a biguanide, chlorohexidine, a triguanide, a bisbiguanide, a polymeric biguanide and a naturally occurring antibiotic compound, as well as analogs, derivatives, salts, ions, and complexes thereof.

A composition optionally includes one or more additives. It is appreciated, however, that a composition is optionally free of an additive. An additive illustratively is one or more antioxidants, anti-static agents, buffering agents, bulking agents, chelating agents, cleansers, colorants, conditioners, deodorants, diluents, dyes, emollients, flavonoids, fragrances, hair conditioners, humectants, ionization agents, moisturizers, occlusive agents, perfuming agents, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. The source and type of additive operable herein is readily understood by one of skill in the art. Illustrative examples of additives are found in WO 2009/090558 and references cited therein each of which are incorporated herein by reference.

A humectant, when included in a composition, helps retain moisture and also prevents rapid evaporation. While it is appreciated that some examples of stabilants may have humectant properties, when a humectant is present in a composition it is in addition to the stabilant and distinguishable therefrom. As such, a humectant optionally does not include glycerin, propylene glycol, ethoxydiglycol, benzyl alcohol, or ethylene glycol when any of these are used as a stabilant. Illustrative examples of humectants include propylene glycol and propylene glycol derivatives, guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, alkoxylated glucose, hyaluronic acid, salts of hyaluronic acid, lactamide monoethanolamine, panthanol, sorbitol, acetamide monoethanolamine and derivatives, esters, salts and mixtures thereof, as well as any suitable humectant found in Handbook of Pharmaceutical Additives published by Gower where one of ordinary skill in the art will recognize suitable humectants contained therein.

Some embodiments of the invention include one or more moisturizers. Examples of moisturizers include allantoin, petrolatum, urea, lactic acid, niacinamide, sodium PCA, shea butter, caprylic/capric/stearic triglyceride, candelilla wax, lanolin, hydrogenated oils, squalene, sodium hyaluronate and lysine PCA. Other examples may be found in the Handbook of Pharmaceutical Additives published by Gower.

Additional additives to benefit skin and its condition include ceramides, glycoceramides, pseudoceramides, sphingolipids such as sphingomyelins, cerebrosides, sulphatides, and ganglioside, sphingosines, dihydrosphingosine, phytosphingosines, and phospholipids. Illustrative examples of ceramides and glycoceramides include those described in U.S. Pat. Nos. 5,589,178, 5,661,118, and 5,688,752. Illustrative examples of pseudoceramides include those described in U.S. Pat. Nos. 5,198,210; 5,206,020; and 5,415,855.

A composition according to the invention optionally has a pH between 1 or less and 8. The pH is optionally at or less than 7, optionally, at or less than 6, 5, 4, 3, 2, or 1.

An inventive composition is optionally provided as a lotion, cream, gel, bar, ointment, or in pad form. Optionally, the composition is provided in a single use container the contents of which are applied directly to the stratum corneum of a subject or applied to an applicator pad for subsequent delivery to the subject.

Also provided is a process of increasing the rate of skin cell renewal in a subject. Skin cell renewal is defined as replacement of a naturally occurring outer layer of dead skin cells with new corneocytes in a stratum corneum. As such, skin cell renewal is distinguishable simply removing the outer layers of skin cells as the process requires maturation of underlying basal cells to outer layer corneocytes. A skin renewal process as it naturally occurs (keratinization) includes movement of basal cells outward from the basal layer, passing through the spinous and granular layers, and eventually becoming corneocytes located in the stratum corneum. The stratum corneum includes approximately 14 layers of dead cells and represents the outer layer of skin cells normally exposed to the environment. Approximately 14 days are normally required in human skin for the basal cells to move from the basal layer to the end of the granular layer and to become corneocytes, and another 14 days for the corneocytes to reach the outermost layer of the stratum corneum. As such, the overall natural skin renewal process takes approximately 28 days. Skin cell renewal rates are increased by the invention from 1% to 150% or more, optionally in excess of 10%, 20%, 30%, 40%, 50%, 60%, 70%, or 80%. Typically, increased concentrations of TCA induce increased skin cell renewal rates, but it is appreciated that skin cell renewal is in the absence of chemical peel.

A process of increasing the rate of skin cell renewal illustratively includes applying a composition as described herein to the skin of a subject. A composition applied to the skin of a subject optionally includes TCA, a stabilant, and optionally, evodiox.

As used herein the term “subject” refers to a human, non-human primate, pig, bovine, equine, mouse, rat, guinea pig, rabbit, hamster, or other mammal.

Application of an inventive composition is appreciated to be topical application. A composition is optionally topically applied at least, once daily, twice daily, or three times daily. In some embodiments, the composition is applied nightly (QHS) such as before a subject goes to bed. The inventive composition is optionally applied weekly, biweekly, or monthly. In some embodiments, a single application of an inventive composition is sufficient to induce increased skin cell renewal in a subject. Typical application times include anywhere from one to 20 weeks or more. It is appreciated that, unlike prior art TCA compositions for topical application, the inventive compositions are suitable for continued use. Continued use is use for an indefinite period of time without interruption. Uninterrupted use (i.e. repeated applications at the same frequency) is possible with the inventive formulations. As such, an inventive process optionally includes uninterrupted use for a period of time. A period as used herein is optionally from one day to 20 weeks or more. A period is optionally indefinite. It is appreciated that while an increase in the rate of skin cell turnover may be observed after as little as a single application, improved results are observed after continued application for a period of time.

The compositions formulated as described herein are optionally topically applied to the skin for the purposes of increasing the rate of skin cell renewal in a volume that will result in application of 0.01 to 10.0 weight percent TCA, optionally 4.0 to 8.0 weight percent. A composition is optionally applied in the areas where fine lines, wrinkles, dry or inelastic skin or large pores are observed. Optionally, a moisturizer is applied with or after application of the inventive compositions to enhance the tactile comfort associated with application of the compositions and to enhance wrinkle effacement and other benefits achieved by the compositions. An improved characteristic of the inventive composition is that the use of additional moisturizers or compositions to reduce the normally observed irritancy of TCA is not required.

In some embodiments, an inventive process includes applying more than one composition of the formulations described herein to the skin of a subject. A second composition is optionally applied simultaneous with, or sequential to a first composition. It is appreciated that a third, fourth, or additional composition is optionally applied simultaneous with or sequential to another composition. A first composition and one or more additional compositions optionally differ by the concentration of TCA present. As an illustrative example, a first composition with a first amount of TCA is applied to the skin of a subject for a first period, and a second composition with a second, differing or equal, amount of TCA is applied to the skin of a subject for a second period. A second or other amount is optionally greater than a first amount of TCA. A first period and a second or additional period are optionally 1 week to 20 weeks or more, or any value or range therebetween. A second period is optionally indefinite. Multiple periods are optionally separated by a rest time. A rest time is optionally one to 30 days or more. A rest period may improve the long term benefit of the applications as it prevents a subject from acclimating to the treatment providing improved results in less overall time.

Also provided is a process of effacing wrinkles in the skin of a subject. Wrinkle effacement is achieved by applying a composition of a formulation described herein to the skin of a subject for a period and thereby effacing the wrinkled appearance of the skin. A composition as formulated herein not only causes increases in skin cell turnover, but also, and optionally in the absence of traditionally known anti-wrinkle agents other than TCA, effaces wrinkles on the skin to which the composition is applied. As such, the modes of administration, periods, and amounts of and elements of a composition of a formulation described herein, optionally in the absence of a non-TCA anti-wrinkle agent, used for increasing the rates of skin cell turnover are operable to efface wrinkles on the skin of a subject.

TCA is considered in the art as unsuitable for use as a wrinkle effacer at concentrations below that of a chemical peel (illustratively 10% or greater) in part due to the high level of irritancy observed with prior art TCA containing compositions. The compositions of the formulations described herein, in contrast, do not suffer from the same level of irritancy such that, for the first time, TCA can be viably used as an agent for wrinkle effacement absent a chemical peel.

Also provided is a process of reducing the rate of TCA degradation in water. In water TCA undergoes decarboxylation to chloroform and carbon dioxide. This process occurs most readily at high temperatures, but will occur spontaneously at ambient temperature. Chloroform is a known carcinogen. (See ACGIH [1994]. 1994-1995 Threshold limit values for chemical substances and physical agents and biological exposure indices. Cincinnati, Ohio: American Conference of Governmental Industrial Hygienists.) Thus, a process is provided for reducing the rate of TCA degradation in water solutions and thereby reducing the carcinogenicity of TCA based compositions. A process illustratively includes combining TCA with a stabilant in an aqueous solution. The combined TCA and stabilant at relative ratios of 0.1:1 to 5:1, optionally 0.5:1 to 2:1 respectively, in an aqueous solution will improve the stability of TCA at 45° C. by 6-fold or more. Any of the compositions of the formulations including a stabilant described herein, and their equivalents, are suitable for the stabilization of TCA.

In some embodiments, TCA stability is increased between 1.5 and 10-fold relative to Aqueous solutions of TCA absent a stabilant. Illustratively, aqueous TCA solutions are stabilized approximately three-fold by glycerin at a TCA:glycerin ratio of 2:1. Adjusting the ratio of TCA:glycerin to 1:2 yields a composition that increases the stability of TCA greater than six-fold.

Various aspects of the present invention are illustrated by the following non-limiting examples. The examples are for illustrative purposes and are not a limitation on any practice of the present invention. It will be understood that variations and modifications can be made without departing from the spirit and scope of the invention. Reagents used are known to those of skill in the art who understand from which sources such agents may be obtained or how such reagents are synthesized from commercially available materials.

### EXAMPLES

### Example 1

Formulating TCA stabilized compositions.

Compositions including TCA at varying concentrations of between 2% to 25% by weight are prepared similarly. All percentages are final composition weight percent. For formation of a 4% TCA based solution with a 1:1 ratio of the stabilant glycerin (Formula A), a stabilant phase is formed including 0.1% xanthun gum dispersed in 4% glycerin at room temperature to create a uniform dispersion. The dispersion is then added to water (55% final w/w) at 70° C. with vigorous mixing using a propeller mixer.

All reagents taught herein are obtainable from standard commercial chemical suppliers known to those of skill in the art, illustratively, Sigma-Aldrich, St. Louis, Mo.

An oil-phase is formed by the combination of cetearyl alcohol and ceteareth-20 (6%) (1:1 ratio sold as PROMULGEN D, Lubrizol Advanced Materials, Inc., Cleveland, Ohio), isopropyl palmitate (6%), cetostearyl alcohol (6%), glyceryl stearate (3%), isododecane (2%), and dimethicone, 350 cst (1%). The oil-phase is then heated to 70° C. An emulsion is created by addition of the oil-phase to the stabilant phase with homogenization at 70° C.

A TCA-phase is formed by the addition of the following ingredients in order—water (10%), TCA (4%), sodium hydroxide (0.9%), evodiox (1%), 1,2-hexanediol and caprylyl glycol (0.5%) (sold as SYMDIOL 68, Symrise, Inc., Teterboro, N.J.), allantion (0.25%), sorbic acid (0.2%), and disodium EDTA (0.05%). The TCA-phase is propeller mixed until uniform and heated to 40° C. It is appreciated that evodiox is the EVODIOX described elsewhere in the specification and is recognized by those of skill in the art as a composition.

The TCA-phase is combined with the emulsion of oil-phase and stabilant phase with mixing under no heating. Mixing is continued while the composition cools until a set-point of 30° C. The resulting formulation is placed into containers and stored at room temperature in jars or airless pump containers.

Formulations B-M are formulated as formula A. Formulas A-M include the following ingredients (final weight percent):

TABLE 1

| | | Formulation (weight percent final) | | | | | | |
|---|---|---|---|---|---|---|---|---|
| | Component | A | B | C | D | E | F | G |
| | water | 55 | 51 | 51 | q.s. to | 55 | 51 | 51 |
| | | | | | 100 w/w | | | |
| | | | | | final | | | |
| stabilant | glycerin | 4 | 6 | 4 | 12.5 | | | |
| phase | propylene glycol | | | | | 4 | 6 | 4 |
| | butylene glycol | | | | | | | |
| | ethanol | | | | | | | |
| | xanthan gum | 0.1 | 0.1 | 0.1 | 0.1 | 0.1 | 0.1 | 0.1 |
| oil | cetearyl | 6 | 6 | 6 | 6 | 6 | 6 | 6 |
| phase | alcohol/ceteareth-20 | | | | | | | |
| | isopropyl palmitate | 6 | 6 | 6 | 6 | 6 | 6 | 6 |
| | cetostearyl alcohol | 6 | 6 | 6 | 6 | 6 | 6 | 6 |
| | glyceryl stearate | 3 | 3 | 3 | 3 | 3 | 3 | 3 |
| | isododecane | 2 | 2 | 2 | 2 | 2 | 2 | 2 |
| | dimethicone, 350 cst | 1 | 1 | 1 | 1 | 1 | 1 | 1 |
| TCA | water | 10 | 10 | 10 | 25 | 10 | 10 | 10 |
| phase | trichloroacetic acid | 4 | 6 | 8 | 25 | 4 | 6 | 8 |
| | sodium hydroxide | 0.9 | 0.9 | 0.9 | q.s. to pH | 0.9 | 0.9 | 0.9 |
| | | | | | 3.0 | | | |
| | evodiox | 1 | 1 | 1 | 1 | 1 | 1 | 1 |
| | 1,2-hexanediol/ | 0.5 | 0.5 | 0.5 | 0.5 | 0.5 | 0.5 | 0.5 |
| | caprylyl glycol | | | | | | | |
| | allantoin | 0.25 | 0.25 | 0.25 | 0.25 | 0.25 | 0.25 | 0.25 |
| | sorbic acid | 0.2 | 0.2 | 0.2 | 0.2 | 0.2 | 0.2 | 0.2 |
| | disodium EDTA | 0.05 | 0.05 | 0.05 | 0.05 | 0.05 | 0.05 | 0.05 |
| | Total | 100 | 100 | 100 | 88.6 | 100 | 100 | 100 |
| | | | Formulation (weight percent final) | | | | | |
| | | Component | H | I | J | K | L | M |
| | | water | 55 | 51 | 51 | 55 | 51 | 51 |
| | stabilant phase | glycerin | | | | | | |
| | | propylene | | | | | | |
| | | glycol | | | | | | |
| | | butylene glycol | 4 | 6 | 4 | | | |
| | | ethanol | | | | 4 | 6 | 4 |
| | | xanthan gum | 0.1 | 0.1 | 0.1 | 0.1 | 0.1 | 0.1 |
| | oil phase | cetearyl alcohol/ | 6 | 6 | 6 | 6 | 6 | 6 |
| | | ceteareth-20 | | | | | | |
| | | isopropyl | 6 | 6 | 6 | 6 | 6 | 6 |
| | | palmitate | | | | | | |
| | | cetostearyl | 6 | 6 | 6 | 6 | 6 | 6 |
| | | alcohol | | | | | | |
| | | glyceryl stearate | 3 | 3 | 3 | 3 | 3 | 3 |
| | | isododecane | 2 | 2 | 2 | 2 | 2 | 2 |
| | | dimethicone, | 1 | 1 | 1 | 1 | 1 | 1 |
| | | 350 cst | | | | | | |
| | TCA phase | water | 10 | 10 | 10 | 10 | 10 | 10 |
| | | trichloroacetic | 4 | 6 | 8 | 4 | 6 | 8 |
| | | acid | | | | | | |
| | | sodium | 0.9 | 0.9 | 0.9 | 0.9 | 0.9 | 0.9 |
| | | hydroxide | | | | | | |
| | | evodiox | 1 | 1 | 1 | 1 | 1 | 1 |
| | | 1,2-hexanediol/ | 0.5 | 0.5 | 0.5 | 0.5 | 0.5 | 0.5 |
| | | caprylyl glycol | | | | | | |
| | | allantoin | 0.25 | 0.25 | 0.25 | 0.25 | 0.25 | 0.25 |
| | | sorbic acid | 0.2 | 0.2 | 0.2 | 0.2 | 0.2 | 0.2 |
| | | disodium EDTA | 0.05 | 0.05 | 0.05 | 0.05 | 0.05 | 0.05 |
| | | Total | 100 | 100 | 100 | 100 | 100 | 100 |

Formula D includes a greater amount of sodium hydroxide to adjust the pH to 3.0. As such the sodium hydroxide and water amounts are q.s. sodium hydroxide to a solution pH of 3.0 and q.s. water to 100% w/w.

The formulations of A-M are further used as a base for the addition of retinol with levels of 0.10 percent hydroxypinnacolone retinoate by weight. When formulations are made using retinols, the retinol, illustratively hydroxypinnacolone retinoate, is added to isododecane to produce a second oil-phase at 40° C. for inclusion in the resulting formulations. The second oil-phase is added to the emulsion followed by homogenization.

### Example 2

Reduction of TCA induced skin irritation. Compositions of formulas N-AA are prepared by the following procedure. Briefly, crystalline trichloroacetic acid (4%) is dissolved in the appropriate volume of deionized water (1 mg/m1). The pH of the TCA is adjusted to a final pH of 3.0 by the addition of sodium hydroxide. After adjusting pH, evodiox alone or along with stabilant is added to the TCA solution with mixing at ambient temperature.

Formulas N-AA are illustrated in Table 2:

TABLE 2

| | Formulation (weight percent-final) | | | | | | |
|---|---|---|---|---|---|---|---|
| Component | N | O | P | Q | R | S | T |
| water | 96 | 95 | 94 | 94 | 94 | 94 | 88 |
| glycerin | | | | | | 2 | |
| propylene glycol | | | | 2 | | | |
| butylene glycol | | | | | 2 | | |
| ethanol | | | 2 | | | | 8 |
| TCA | 4 | 4 | 4 | 4 | 4 | 4 | 4 |
| evodiox | | 1 | | | | | |
| | Formulation (weight percent-final) | | | | | | |
| Component | U | V | W | X | Y | Z | AA |
| water | 88 | 88 | 88 | 91 | 91 | 91 | 91 |
| glycerin | | | 8 | | | | 4 |
| propylene glycol | 8 | | | | 4 | | |
| butylene glycol | | 8 | | | | 4 | |
| ethanol | | | | 4 | | | |
| TCA | 4 | 4 | 4 | 4 | 4 | 4 | 4 |
| evodiox | | | | 1 | 1 | 1 | 1 |

Each of formulas N-AA are tested for irritation on human skin in 28 volunteers. A pain response (stinging) test is performed by applying 0.1 ml of the compositions of formulas N-AA to the nasolabial fold region. This test site that is highly innervated with sensory fibers. The pain response test is an established method for interrogating dermatological substances for their propensity to induce irritation. As a control, saline solution is applied in equal volume to the contralateral test site. The compositions are allowed to incubate on the skin for 1 minute and an overall pain response is recorded on a scale of 1 to 5 with 1 equating to no increase in pain or other sensitivity relative to control side, and 5 equating to severe pain or other sensitivity relative to control. The results for each of the formulations are presented in Table 3.

TABLE 3

| Formula | Irritation |
|---|---|
| N | 5+ |
| O | 3.8 |
| P | 5+ |
| Q | 4.8 |
| R | 4.5 |
| S | 4.4 |
| T | 5+ |
| U | 4.2 |
| V | 4.2 |
| W | 4.1 |
| X | 3.9 |
| Y | 2.7 |
| Z | 2.6 |
| AA | 2.4 |

The solution with TCA alone induces a high level of irritation. The addition of the stabilant ethanol at 8% or 2% fails to reduce the irritation level. The addition of evodiox either in the absence of stabilant or along with ethanol successfully reduces irritation of the TCA to 3.8 or 3.9, respectively, indicating the ability of the evodiox alone to reduce the irritability of TCA. In formulations with the stabilants glycerin, propylene glycol, or butylene glycol, the level of irritation is reduced with the greatest reductions in irritation found at 8% stabilant. The presence of both evodiox and a stabilant (non-ethanol) synergistically reduce irritation of the TCA compositions to highly tolerable levels with scores of 2.7 or below.

The irritation of formulas N-AA are also assessed using a Chroma Meter (Konica Minolta). Two test sites are chosen on the cheeks of 28 human volunteers for testing of a TCA composition or saline control. Each test site is approximately 3 centimeters in area. For each test site 1 ml of formulas N-AA or saline are applied to the test site and allowed to absorb for 20 minutes. The degree of redness is measured at the test and control sites 25 minutes after application. The results are similar to the pain response test with a synergistic reduction in irritation by the combination of a non-ethanol stabilant and evodiox.

The pain response test and the Chroma Meter test are repeated with compositions of formulas A-C and E-M. Each formulation shows reduced irritancy relative to the TCA solution of formula N. Each of the formulations of A-C and E-M show greater relative irritancy reduction compared to TCA solutions with ethanol alone or evodiox plus ethanol alone. The greatest level of relative irritancy reduction is observed with formulations A-C and E-J.

### Example 3

TCA-stabilized compositions increase wrinkle effacement in human skin. To assess the ability of TCA-stabilized compositions for their ability to reduce wrinkles in human skin, two additional formulations are prepared with the composition of Table 4. Compositions BB and CC are prepared as in Example 1.

TABLE 4

| | | Formulation | |
|---|---|---|---|
| | Component | BB | CC |
| | water | 59 | 53 |
| stabilant | glycerin | 2 | 5 |
| phase | xanthan gum | 0.1 | 0.1 |
| oil phase | cetearyl alcohol/ceteareth-20 | 6 | 6 |
| | isopropyl palmitate | 6 | 6 |
| | cetostearyl alcohol | 6 | 6 |
| | glyceryl stearate | 3 | 3 |
| | isododecane | 2 | 2 |
| | dimethicone, 350 cst | 1 | 1 |
| TCA phase | water | 10 | 10 |
| | trichloroacetic acid | 2 | 5 |
| | sodium hydroxide | 0.9 | 0.9 |
| | evodiox | 1 | 1 |
| | 1,2-hexanediol/caprylyl glycol | 0.5 | 0.5 |
| | allantoin | 0.25 | 0.25 |
| | sorbic acid | 0.2 | 0.2 |
| | disodium EDTA | 0.05 | 0.05 |
| | Total (weight percent) | 100 | 100 |

Thirty human subjects, 4 males and 26 females (age range: 18-65 years) are included in a three month trial with follow ups at 6 and 12 weeks. Wrinkle effacement is measured by both a self rating assessment scale and using a Visioscan VC98 (Courage+Kahazaka, Koln, Germany) for objective skin surface evaluation. The self rating system is on a scale of 0 to 3; 0=no change; 1=mild change; 2=moderate change, 3=extreme change.

All subjects apply the test formulation BB on a site of winkles on the face nightly for the first six weeks. After the initial 6 week evaluation, the subjects are switched to formulation CC for the final 6 weeks when a final evaluation is made. Subjects are allowed to use sunscreens and cleansers of their choice during the test period, which varies among the subjects. Prior to beginning the test regimen and at both the initial and final evaluations, all subjects are photographed and the area to which the formulations are applied are analyzed by the Viscoscan system.

The study results are presented in Table 5.

TABLE 5

| | Evaluation | |
|---|---|---|
| | 6 | 12 |
| Characteristic | weeks | weeks |
| Softer texture | 1.5 | 1.8 |
| More even color | 1 | 1.5 |
| Improved skin glow | 1 | 1.6 |
| Smaller pores | 0.5 | 1.4 |
| Reduced wrinkles | 0.7 | 1.3 |
| Firmer skin | 0.8 | 1.7 |
| Overall improvement | 1.2 | 1.8 |

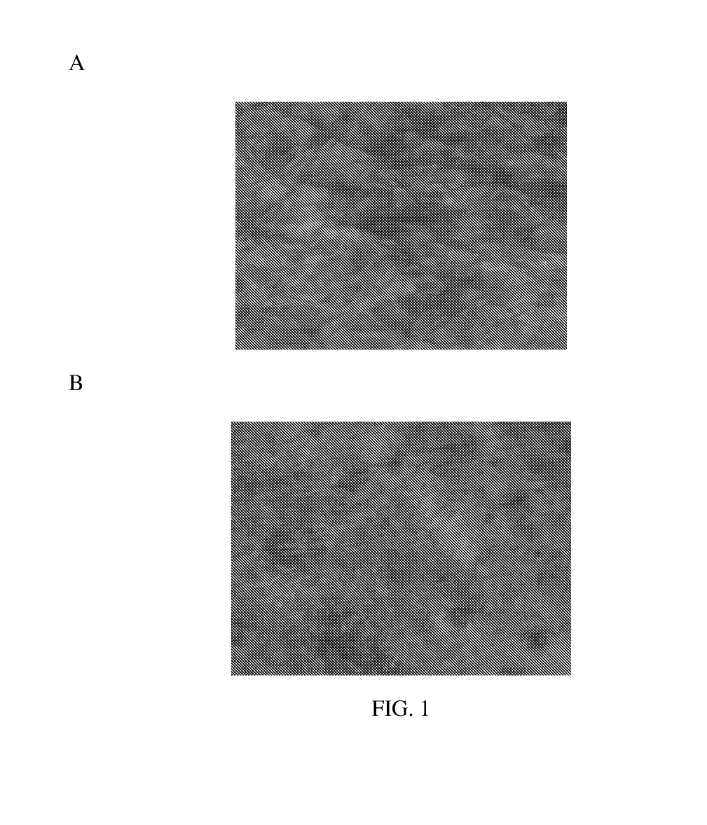

Overall, subjects report progress toward improved skin characteristics with significant steps at 6 weeks in texture, color, and glow, as well as improvement in wrinkle reduction and firmness. At 12 weeks of use all subjects report improvement in all test parameters with additional significant improvements in smaller pores, reduction in wrinkles, and firmer skin. An illustrative improvement in skin wrinkles is illustrated for one subject (patient J.B.) in FIG. 1 demonstrating wrinkles before treatment (A) and after 12 weeks of treatment (B).

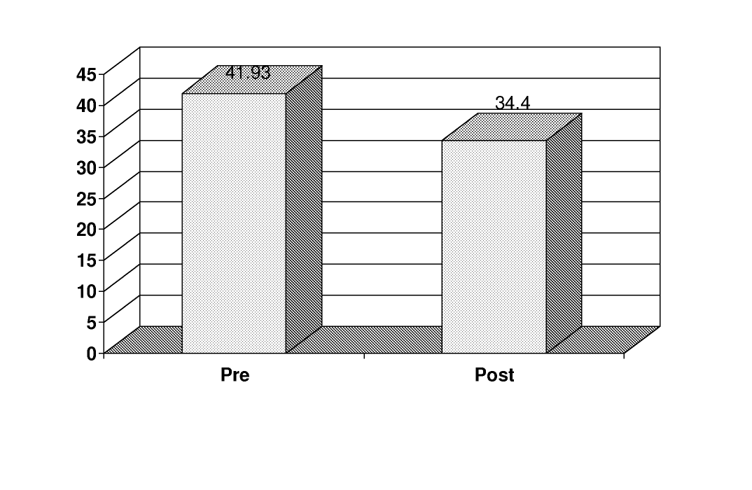

The test subjects are also evaluated with the Visioscan system. At the 12 week evaluation, an average reduction in wrinkles of 22.9% (range 0% to 40.5%) is observed. The quantitative wrinkle reduction (Se_{w}) for patient J.B. is illustrated in FIG. 2 and corresponding to level of wrinkle effacement demonstrated in the photograph of FIG. 1. The skin of all subjects shows a reduction in skin roughness (Seᵣ) of 35.6% (range 11% to 62.5%). Thus, the TCA-stabilized composition formulations BB and CC increase wrinkle effacement during a short, regular application period.

The formulations of A-M, and Q-S and U-AA are also tested and show both reduction in wrinkles at 6 and 12 weeks.

### Example 4

Enhanced skin cell renewal by TCA-stabilized compositions. The compositions of formulations A-CC are tested for their ability to enhance skin cell turnover relative to saline (control). Human volunteers are tested for changes in skin cell turnover by the methods of Smith, WP, Cosmetics and Toiletries, 1994; 109:41-48. Briefly, the dansyl chloride method is used where dansyl chloride that is milled into petrolatum USP at 5% is applied under an occlusive patch to forearms for a period of 24 hours. Staining of skin cells is measured by quartz mineral lamp. The compositions of formulations A-CC are applied to the test sites once per day (evening). The reduction in staining is monitored by daily measurements under quartz lamp.

Subjects are also measured for exfoliation rate by a forced exfoliation method. Briefly, skin squames are collected by adherence to a tape coated microscope slide pressed into the skin surface and quickly removed. The number of cells is measured by opacity.

Each of formulations A-BB show increased rates of skin cell turnover as measured by both the dansyl chloride method and the forced exfoliation method by 10 to 80% with greater amounts of TCA producing greater rates of skin cell renewal. The rate of skin cell renewal is increased with increasing concentration of TCA in the formulation. Formulations including a non-ethanol stabilant, evodiox, or both also report little to no irritation during the test period relative to subjects that apply formulas N, P, and T who each report significant irritation of the test sites.

### Example 5

Stabilization of TCA in aqueous solutions. The ability of stabilants and evodiox to reduce or maintain TCA levels in aqueous solutions is measured essentially as described by Dreshsel, D. et al, Chromatographia, 2001; 54:151-154 for the measurement of chloroform liberated from TCA at high temperatures. Test formulations N-AA are tested for the level of TCA prior to and following a storage period. Each formulation is stored in a sealed container at 45° C. for a period of 90 days and the level of TCA reductions in the material are determined.

Briefly, A Hewlett-Packard 5890 II gas chromatograph is used to measure chloroform liberated from TCA. Compounds in the formulations are separated from other components of the formulations by binding to a 30 cm×0.32 mm inner diameter CP-SilicaPLOT column (Varian-chrom-pack) with hydrogen carrier gas. Column temperature is held at 30° C.

A PTV injector equipped with a glass liner packed with Tenax TA is used to adsorb the TCA during injection. Initial injection temperature is 50° C. after which the injector is heated at 12° C./sec to 130° C. for a 5 minute hold with the split valve open to remove volatile compounds. The injector is then heated at 12° C./sec to a final temperature of 250° C. for 5 minutes with the split valve closed to fully decarboxylate the TCA. The split valve is then opened and the sample injected into the instrument.

The procedure is functional as the volatile CO₂ and CHCl₃ are removed prior to decarboxylation of the TCA such that no appreciable contamination from previously liberated CHCl₃ during the storage decarboxylation of TCA occurs. As such, the TCA measurements obtained from the instrument are representative of the intact TCA remaining in the formulations after the storage period.

The percent losses of TCA after the 90 day storage period at 45° C. relative to the amount of TCA prior to storage (immediately after production of the formulation) are presented in Table 6.

TABLE 6

| | % TCA |
|---|---|
| Formula | Loss |
| N | 12.6 |
| O | 12.3 |
| P | 7.1 |
| Q | 5.4 |
| R | 4.3 |
| S | 4.2 |
| T | 4 |
| U | 2.2 |
| V | 2.1 |
| W | 2 |
| X | 4.1 |
| Y | 2.5 |
| Z | 2.4 |
| AA | 2.3 |

The presence of TCA alone in an aqueous solution degrades by 12.6% over the 90 day storage period. Ethanol at 2% or 4% modestly improves the stability of TCA (see Formulas P and T). The stabilants glycerin, propylene glycol, and butylene glycol each with lower volatility than ethanol considerably improve the stability of TCA by 6-fold or more. This increase in TCA stability is due to the stabilant and cannot be attributed to the presence of evodiox as evodiox alone does not appreciably alter the stability of TCA (e.g. compare formula N with O, and W with AA).

The study is repeated with formulas A through M, BB, and CC. While all formulations improve the stability of TCA, the formulations with glycerin, propylene glycol, or butylene glycol as stabilants are superior to ethanol.

Thus, the presence of stabilant effectively improves TCA stability in aqueous solutions indicating that lower amounts of chloroform will be present in stored TCA solutions by the addition of such stabilants.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified. Methods of nucleotide amplification, cell transfection, and protein expression and purification are similarly within the level of skill in the art.

Patents and publications mentioned in the specification are indicative of the levels of those skilled in the art to which the invention pertains. Each patent, application or publication is incorporated herein by reference to the same extent as if each individual patent, application or publication was specifically and individually stated as incorporated herein by reference for the specific teaching for with each reference is cited and additionally for entirety of their contents including text, figures, and references.

The foregoing description is illustrative of particular embodiments of the invention, but is not meant to be a limitation upon the practice thereof. The following claims, including all equivalents thereof, are intended to define the scope of the invention.

1. A composition for topical administration comprising:
trichloroacetic acid present at less than 10% by weight;
a stabilant with a normal boiling point in excess of 50 degrees centigrade;
said trichloroacetic acid and said stabilant present in a ratio from 0.1:1 to 5:1.

2. (canceled)

3. The composition of claim 1 wherein said trichloroacetic is present at or below 5 percent by weight.

4. The composition of claim 1 further comprising an anti-irritant.

5. The composition of claim 4 wherein said anti-irritant comprises one or more indolequinazoline alkaloids.

6. The composition of claim 4 wherein said anti-irritant is evodiox.

7. The composition of claim 4 wherein said anti-irritant is present at 0.1 percent to 5 percent by weight.

8. The composition of claim 1 wherein said stabilant is propylene glycol, butylene glycol, ethoxydiglycol, benzyl alcohol, or glycerin.

9. The composition of claim 1 further comprising a bioactive agent.

10. The composition of claim 9 wherein said bioactive agent is: vitamin A or a derivative of vitamin A; vitamin E or a derivative of vitamin E; a hydroxy acid; benzoyl peroxide; resorcinol; an antimicrobial; an anti-neoplastic agent; an anti-viral agent; a nonsteroidal anti-inflammatory agent; a UV filter; or an immunomodulator.

11. The composition of claim 10 wherein said agent is vitamin A or its derivatives wherein said vitamin A or its derivatives are present at 0.001 to 2 weight percent.

12. The composition of claim 11 wherein said vitamin A derivative is: retinal; retinoic acid; retinyl ester; retinol; tretinoin; isotretinoin;
adapalene; tazarotene; or combinations thereof.

13. The composition of claim 10 wherein said hydroxy acid is salicylic acid, acetylsalicylic acid, or combinations thereof.

14. The composition of claim 10 wherein said agent is vitamin E or a derivative of vitamin E wherein said vitamin E or derivative of vitamin E is present at 0.1 to 10 weight percent.

15-21. (canceled)

22. A process of preventing decarboxylation of trichloroacetic acid in water comprising:
combining trichloroacetic acid with water; and
further combining a stabilant with a normal boiling point in excess of 100 degrees Celsius.

23. (canceled)

24. (canceled)

25. A process for increasing wrinkle effacement comprising:
applying a first composition that is the composition of claim 1 to the skin of a subject at a site of a wrinkle for a first period.

26. (canceled)

27. The process of claim 25 further comprising applying said composition daily for a period of twenty days or more.

28. The process of claim 25 comprising applying said composition daily and wherein said first period is 6 weeks or more.

29. (canceled)

30. The process of claim 25 wherein said composition comprises 4% trichloroacetic acid by weight applied nightly and wherein said first period is 6 weeks.

31. The process of claim 25 wherein said composition comprises 5 percent trichloroacetic acid.

32. The process of claim 25 wherein further comprising applying a second composition that is the composition of claim 1 and having a greater concentration of TCA than said first composition.

33-35. (canceled)
